(19) United States
(12) Patent Application Publication
(10) Pub. No.: US 2020/0013498 A1
(43) Pub. Date: Jan. 9, 2020

(54) Method for Adjudicating One or More Health Care Service Bids

(71) Applicant: Arthur Morris Gelber, Ridgefield, CT (US)

(72) Inventor: Arthur Morris Gelber, Ridgefield, CT (US)

(21) Appl. No.: 16/501,815

(22) Filed: Jun. 11, 2019

Related U.S. Application Data
(63) Continuation-in-part of application No. 15/731,087, filed on Apr. 17, 2017.
(60) Provisional application No. 62/323,409, filed on Apr. 15, 2016.

(51) Int. Cl. G16H 40/20; G06Q 30/08; G16H 40/40; G16H 10/60

(52) U.S. Cl. CPC G16H 40/20; G16H 10/60; G16H 40/40; G06Q 30/08

(57) ABSTRACT

The present invention is an intermediary through which health care services are purchased by the intermediary at wholesale prices from the health care system and auctioned by the intermediary via a distribution channel. Financial transactions are simultaneously settled through the invention via arbitrage. The intermediary is not a managed care organization, insurer, or reinsurer with respect to health insurance. It accepts financial risk and pays for the delivery of health care services. The present invention provides a method of receiving via the graphical user interface (GUI) a user selection of health care service(s) coupled with an offer of a price for a health care service. Determining, via a processor, the qualifying circumstances and matching reference data, whereby subjecting financial transactions to adjudication; consequentially lowering health care cost without compromising quality of health care.

### BACKGROUND

Based on a recent study, 2018, more U.S. adults are under insured compared to 2015 with the largest growth among people with job-based health plans. Twenty-eight percent of U.S. adults who have insurance through their employer were underinsured in 2018, up 20% just four years earlier. At the same time, people who bought plans on their own through the individual market or marketplaces were most likely to be underinsured with 42 percent reporting a lack of adequate coverage in 2018. Under insured adults reported having trouble affording their health care; they are having problems getting care, 41 percent of underinsured adults said they delayed needed care because of cost, compared to 23 percent of people with adequate insurance coverage. Almost half (47%) of underinsured adults report medical bill and debt problems—nearly twice the rate as those who were not underinsured (25%) In 2018, 61 percent of people who reported having a gap in their insurance said they had been without coverage for less than six months, compared to 31 percent who had been uninsured for a year or longer.

The health care economy is slowing seeing some adjustments market the growing awareness of the wide variation in health care prices, increased availability of price data, and increased patient cost sharing are expected to drive patients to shop for lower-cost medical services. In a recent survey, most respondents believed that price shopping for care is important and did not believe that higher-cost providers were of higher quality. Common barriers to shopping included difficulty obtaining price information and a desire not to disrupt existing provider relationships.

### Rates of Price Shopping for Care

Fifty-two percent of the respondents were aware of the price before they received care, and 13 percent had searched for their expected out-of-pocket spending. Ten percent reported that they had considered going to another provider, and 3 percent had compared costs across providers. Of the 13 percent who had actively searched for out-of-pocket spending, 63 percent had called their provider for information, 25 percent reported using a website sponsored by their health plan or employer or a website available to the public, and 9 percent had called their health plan directly (data not shown).

To help patients identify lower-cost providers, price transparency tools are now available through health plans; employers or public, state-level sites Over half of the states have passed laws or regulations encouraging either payers or providers to disclose pricing information to patients.

The method of adjudicating health care service bids by example: Table 1, 2, 2, illustrated a 28% saving to the user of this inventive process. There are over 10,000 procedural codes, and more than 15,000 reasons for seeking health care services, with millions of possible computations before selecting a health care service. One intent of the method is to leverage technology, make us of data streaming, information technology, rules based engine technology, and to put forth the information in simple to understand language, so that the user of the web-enabled device can make an informed decisions when making a bid for a health care service.

### BRIEF SUMMARY OF THE INVENTION

The present invention is an intermediary through which health care services are purchased by the intermediary at wholesale prices from the health care system and auctioned by the intermediary via a distribution channel. Financial transactions are simultaneously settled through the invention via arbitrage. The intermediary is not a managed care organization, insurer, or reinsurer with respect to health insurance. It accepts financial risk and pays for the delivery of health care services. The present invention provides a method of receiving via the graphical user interface (GUI) a user selection of health care service(s) coupled with an offer of a price for a health care service; wherein, determining, via a processor, qualifying circumstances that are predefined by reference data, subjecting financial transactions to adjudication; adjusting one or more bids, whereby lowering health care cost for the user without compromising quality of health care.

In one embodiment, programmatically, a determination, via a processor, identifies qualifying circumstances wherein predefined reference data will be used to systematically adjudicate the user's health care service selection(s) and adjoining bid(s), whereby the majority of adjudicated financial transaction result in a reduction of the monetary value of the user's original successful bid.

In some embodiments, receiving, via a graphical user interface (GUI) of a computer, one or more user selections of health care service(s) with an adjoining user bid(s), an automated process takes the user data, extracts the information required for adjudication, transforms it into a format that can serve its business needs, and loads it to a centralized data repository, such as a centralized data warehouse and/or an in-memory-database management system.

Some embodiments include, a streaming data architecture provides the means for receiving and sending data streams and executes system applications with real-time analytics logic. Upon receiving a user query, the stream processor reacts in real- or near real-time and triggers an action, such as remembering the process event for future reference.

Some embodiments include, a shift from a monolithic architecture like a central data warehouse to a decentralized to one built with microservices. Each microservice is a cache comprised of a root table and a primary key and a primary key can be linked to a plurality of data source such as reference data protocols. Reference data is a means to identifying qualifying circumstances that trigger the adjudication of health care services and adjoining bids throughout the invention. The streaming data architecture co-locates data processing with storage to lower application response times (latency) and improve throughput. In terms of the invention, it offers an overall improvement to data processing, and ultimately to the user's experience.

Some embodiments, each cache runs a unique process and usually manages its own database. Each unique process can represent a plurality of conditions and each condition can have its own set of unique reference data protocols, and protocols can be mapped to HIPAA data set codes, medical terms, clinical terms, synonyms, clinical polices, standards of care, and any of these cross-walks can be linked to age, gender, and population demographics. For instance, a surgical cache may include various conditions or reasons to reduce the monetary value of the originating successful bid for the user. Receiving, via the GUI, a user selection of multiple surgical procedures, and the user scheduled dates of services for two or more surgeries, at a hospital on the same day, the surgical cache would matched the primary surgical CPT codes to one or more indicators that are represented another table header as “indicator” and retrieve the indicator to determine, by a processor the type of indicator, and pairing CPT codes to indicators to reference data protocols. Processing, multiple surgery rules, as follows: applying 100% of the user's highest valued surgical procedure selection, and 50% for any additional surgical procedures. Receiving, via a GUI on a computer system, the user from a widget on the landing page can view adjudication results from an invoice generated from a computer.

In another embodiment, the present invention is an intermediary through which health care services are purchased by the intermediary at wholesale prices from the health care system and auctioned by the intermediary via a distribution channel. Financial transactions are reconciled and settle via arbitrage.

In another embodiment, from a widget on the landing page, via a GUI, the bidder is asked to provide certain identification if he/she is an existing user, or to register as a new user. This is followed by the user selecting one or more health care services coupled with a bid for the desired health care service.

In some embodiments, the method disclosed herein may include providing a module, the module being a data loading module for loading data into a target database that is associated with a data integration platform;

In another aspect, the invention may transform data and deliver it throughout the system, with a focus on processing data in motion, wherein treating extract-transform-load (ETL) as batch processing, as just one or more actions in a continuous stream of user events, reducing the delay before a transfer of data begins following an instruction for its transfer; whereby improving the performance and user experience.

In another aspect, of the invention, the system receives and sends data streams and executes the applications, via a stream processor, wherein said processor reacts in real- or near real-time and automatically triggering an action, such as adjudicate, and store the event for future reference. In another aspect, of the invention, it may adjudicate the health care service(s) and bid(s), determining, via multiple processors various conditions, automatically subjecting user bids to a mapping process, that include the steps of targeting data in primary and/or secondary storage facility, identifying qualifying circumstances, joining qualifying events to reference data protocols, adjudicating user bid(s) based on stored instruction.

In embodiments of the invention, the financial and adjudicating event-driven processing triggers may initiate one or more actions, such as triggering the auction module, for example: If the adjudicated bid(s) price exceeds the formulated internal threshold price, the transaction then occurs at the adjudicated bid price. If the adjudicated bid price is below the formulated internal threshold price the invention informs the user, via the GUI, that his/her bid is too low and then the user is given the opportunity to submit an incremented bid and the comparison with the formulated internal threshold price is done again.

In one embodiment, the invention may be determining, via a processor a formulated internal threshold above which it will accept the bid from the user. As the user bid(s) succeeds, there may be two revenue models. The first is the difference between the user's bid(s) and the formulated internal threshold price and the second is the difference between the formulated internal threshold price and the health care provider's registered wholesale prices.

In another embodiment, if the financial transaction is successfully completed the user the user may schedule an appointment for a preferred date of service with their designated health care provider or supplier or both. The messaging module will send text messages, emails, to the user and the provider of health care services. The provider of health care services receives a letter of guarantee of payment with a list of services purchased by the user and a confirmation of the date of service(s). Additionally, a remote third-party vendor will send out paper invoices to the user with a randomly assigned confirmation alpha-numeric number, which may include in invoice with a list of health care services, the scheduled date of service, and the contact information for the provider of health care services. The next step, scheduled email and voice messages will be triggered between the date of purchase and the 24 hours before the schedule date of service.

In another embodiment, if, from the purchase date of the health care service to 24 hours before the scheduled date of service, the electronic messages module will generate an electronic “Price Alert” to a user's web enabled device or fax number when a promotional offer or a lower price is available within 25 miles from the home resident of the user before the date of service, a “price guarantee” to the user.

In one embodiment the invention method may include a search box, a widget on the landing page for searching one or more health care services, the method comprising of the steps of receiving, via the GUI, a user selection for one or more health care services, wherein the user selection(s) are processed by a translation engine and converted into one or more HIPAA data set codes, such as a CPT code, wherein the receiving and sending of data streams, executing the application are part of the stream data architecture. There are multiple streaming processing tools available today, Apache Samza, Apache Storm, and Apache Kafka. These streaming tools can be used to shift from a centralized data repository to a decentralized grouping of adjudication caches, whereby lowering application response times-latency and improve throughput. Upon receiving the user queries, the stream processor can react in real- or near real-time and triggering one or more actions, such as remembering the details of the user's financial transactions for future reference.

In another embodiment, the invention may be a framework for expediting user searches for health care service searches and adjudicating financial transactions on a GUI; from a technology prospective, the user data is transferred and processed from source databases and mapped to the IMDB adjudication. The IMDB adjudication aggregates data from the one or more cache groups, transforms the data, matches the primary key to parent-child relationships, determining, via a processor targeted data sources, mapping ETL data to reference data, matching parent and child data relationships to the reference data and coupling reference data protocols to define qualifying circumstances, and repricing or adjusting qualified bids based on the source data criteria. The IMDBS adjudication streaming processor transformation raw data to achieve a data format to minimize data upload errors and being able to perform analytics and reporting.

In some embodiments, the invention comprises of steps for targeting reference source data, identifying indictors under table headers and joining primary key-rows of CPT codes, combining multiple databases where the primary key match to qualifying indicators and qualifying indicators are one of many indicators that define a qualifying circumstance, and the qualifying circumstances triggers an action which is consistent with the source data protocols.

In one embodiment, the invention is a method for searching on a landing page from a widget for a health care service, the method comprising of receiving, via a GUI a user selection of one or more health care services.

In another embodiment, the invention is a method of receiving, via a GUI, a semantic search for a health care services and using a translation engine processor to convert the semantic search to one or more HIPAA code sets, like CPT codes. The user can search for a health care service(s) via semantic search, inputting one or more of the HIPAA code sets data, either the user input is subject to translation or an extract-transform-load (ETL) protocol.

In another embodiment, the user may prefer selecting a health care service(s) via the GUI of a computer system, accessing a virtual scribe. From a widget on the landing page of the user's web enabled device, the user inputs the chief complaint, and some associated common symptoms. In another embodiment, the semantic information is processed by the translation engine, the ETL functions, extract, transform, load are combined to pull data out of cache a plurality of source data, transforming multiple data sources, targeting data multiple tables, determining, via a processor data sources with clinical terms, medical terms, synonyms, including HIPAA code set databases. Multiple processors may be used to expedite the process, one such process may be a streaming processor. The data integration begins the process of combining data from different data sources to a single, cohesive data set. The IMDB adjudication aggregates all the data, including information stored in the cache. The findings are transformed in understandable terms for the user to understand. The user may proceed to select, via the GUI of a computer the health care service from a list of informative options.

In another embodiment, the invention is a system, where each adjudication cache has a root table comprising a section of the HIPPA data set code, one such example would be a CPT code database. A CPT code database includes approximately 10,000 CPT codes, with short description, that contains a group of CPT codes which for this illustration is the primary key. A subset of the primary key is the child relationship, where each child relationship to the primary key represents a section of the medical code set; the evaluation and management header is linked to the primary key CPT code rows range from 99201-99499; Anesthesia header has a rows of CPT codes ranging from 00100-01999; 99100-99150; Codes for surgery header range from 10000-69990; codes for radiology header range from 70000-79999; Codes for pathology and laboratory header range from 80000-89398; and Codes for the header medicine range from 90281-99099; 99151-99199; 99500-99607. Furthering this illustration, each subsection and range of primary keys are stored in an adjudication cached. Each of the Adjudication caches include guidelines that are unique to that section. These directions are not all inclusive or nor limited to, definition of terms, modifiers, unlisted procedures or services, special or written reports, details about reporting separate, and multiple or starred procedures and qualifying circumstances. Some of the CPT codes are date sensitive, some of the improper coding relationships include but are not limited to unbundling of health care, incidental health care services, mutually exclusive ser

Some user selected health care services may represent improper health care services codes. Examples of improper coding categories include but are not limited to unbundling, which is the user making a bid on one health care service that is incidental or included in a primary or more comprehensive service; another example, the user bids on a surgery without knowing there may be some hidden charges, such as paying for a co-surgeon during the surgical procedure; Another example, certain rules and edits apply for a subset of surgical procedures, if those surgical procedures are performed on the same day. In one embodiment of the invention, if more than one surgical procedure is coupled with a user bid, a determination, by a processor will identify the surgical procedure with the highest bid and allow the bidder to purchase that surgical procedure at 100% of the bid price, however, the second surgical procedure will be adjusted, whereby the user's bid will be reduced by 50 percent, if there is a third surgical procedure the invention will reprice the third procedure whereby discounting the third and so on down by 50 percent. There is a plurality of databases stored in the central databases, a subset of those databases is stored in the adjudication in-memory database. The reference data or the reference source data that initially defined the source date should be based on American Medical Association (AMA) CPT® guidelines, CMS guidelines, Correct Coding Initiatives, HIPAA data set codes, medical societies, and other widely accepted third-party resources. All guidance should change over time, and source edits and rules should be kept up to date. The source data should automatically be reviewed for edits and rules errors and questionable coding relationships by testing the data against an expansive knowledgebase containing millions of government and industry rules that cover health care claims.

In yet another embodiment, the invention is a method to combine qualify circumstances and reference data protocols to adjudicate health care service and adjoining bid(s).

### BRIEF DESCRIPTION OF THE DRAWINGS

FIG. 1 shows an illustrative embodiment of a central data warehouse and in-memory database adjudication, backend and front-end in accordance with the present disclosure.

FIG. 2 shows an illustrative embodiment of input devices sending messages to a computer system for processing, and an output device reproducing or displaying the results of the processing.

FIG. 3. Shows an illustrative embodiment of a process flow for adjudicating and adjusting an example of reference data and protocols

FIG. 4 Illustrates a dataflow of the invention

### DETAILED DESCRIPTION

As used herein, “knowledgebase” containing millions of government and industry edits and rules that cover health care claims or “data” “source data” or “data source” or “data targets” or “reference data” are intended to have the broadest possible meaning consistent with the terms, and shall include a database, a plurality of databases, a repository information manager, a queue, a message service, a repository, a data facility, a data storage facility, a data provider, a website, a server, a computer, a computer storage facility, a locally connected computing facility, a remotely connected computing facility, a client, a laptop a personal digital assistant, a telephone, a cellular phone, a mobile phone, an information platform, a processing facility, intermediary platform or other facility where data is handled or other facility provided to store data or other information, as well as any files or file types for maintaining structured or unstructured data used in any of the above systems, or any streaming, message, event driven processing, or otherwise sourced data, and any combination of the foregoing, unless a specific meaning is otherwise indicated or the context of the phrase requires otherwise.

“Storage” or “stored” data as used herein, is a cache mechanism which may be any logical or physical device, resource, or facility capable of acting as a data source or data target.

“Arbitrage” as used herein, is the ability to simultaneously buy and sell a health care service based on price discrepancy.

“Adjudication”, as used herein, is the act or process of applying health care system edits and rules to the purchasing of health care services rendered by a provider(s)/supplier(s) to one patient for an episode of care.

“Adjudication Cache”, as used herein, include data source definitions that are associated with data schema.

“Bid” or “bids” “offer” “offer price” “tender” “proffer” as used herein, is an offer of a price for one or more health care services by a user.

“Bidding Context”, as used herein, is the circumstance that formed the setting for an event, statement, or idea, like making an offer for a health care service

“Central data warehouse” or “Central Repository” or “data warehouse” or “centralized” data warehouse, enterprise data warehouse, as used herein, is a repository of source data, knowledgebase data, reference data, transactional data, target data, business logic, where one or more databases are created from operation data extracts.

“Data Mart” or “Adjudication Cache”, as used herein, is generally understood as comprising a partition of the centralized data repository (primary storage).

“Data base manager” or “Adjudicator” or “Adjudication Manager” as used herein, is a computer program, or a set of computer programs, is specialized system software for creating, managing databases, and provides controlled access to the data. It uses a tabular structure where the headers are in column names, and the rows contain corresponding values.

“Event” or “Event driven” as used herein, are user actions, sensor outputs, or messages; Programmatically, it defines the action the program should take when the event occurs.

A “Financial transaction(s)” as used herein, is an agreement, or communication, carried out between a buyer and a seller to exchange a health care service for a negotiated price.

“Distribution Channel”, as used herein, may be a direct or indirect distribution channel, it allows the user to buy health care services and products from an intermediary. The primary purpose of any channel of distribution is to bridge the gap between health care providers and suppliers and users of it whether the parties are in the same community or in different countries thousands of miles apart.

“Health Care System” or “health system” or healthcare system, as used herein, means is the organization of people, institutions, and resources that deliver health care services to meet the health needs of target populations.

“Intermediary platform” as used herein, is a combination of technical and business processes used to combine disparate sources into meaningful and valuable information. The Intermediary platform allows data from multiple sources to be collected, sorted, and transformed so that it can be applied to various business transactions or routed to specific users, business units, partners, applications, or prospective solutions.

As an intermediary the invention offers intermediation services between its trading parties. The intermediary buys health care services directly from the health care system. The intermediary is not a managed care organization, insurer, or reinsurer with respect to health insurance. Financial transactions are reconciled through the intermediary via arbitrage. The invention pays for authorized invoices for the delivery of health care services, accepts financial risk for the delivery of health care services and establishes, operates or maintains an arrangement or contract with health care providers and suppliers relating to (A) the health care services rendered by the providers, and (B) the amounts to be paid to the providers for such services.

“Health care services” means health care related services or products rendered or sold by a provider within the scope of the provider's license or legal authorization, and includes hospital, medical, surgical, dental, vision and pharmaceutical services or products.

“HIPAA Data Set Codes”, as used herein, is any set of codes used for encoding data elements, such as tables of terms, medical concepts, medical diagnosis codes, or medical procedure codes. Medical data code sets used in the health care industry include coding systems for diseases, impairments, other health related problems, and their manifestations; causes of injury, disease, impairment, or other health-related problems; actions taken to prevent, diagnose, treat, or manage diseases, injuries, and impairments; and any substances, equipment, supplies, or other items used to perform these actions. Code sets for medical data are required for data elements in the administrative and financial health care transaction standards adopted under HIPAA for diagnoses, procedures, and drugs.

“in-memory database (IMDB or database management system (IMDBMS)”, as used herein, is an application where response time is crucial. IMDB primary depends on main memory for storing computer data. IMDCs are quicker than disk optimization because they carry out few CPU instructions, and their internal optimization algorithms are much simpler. IMDB eradicates disk access by saving and manipulating data in the main memory.

“Knowledgebase” or “Knowledgebase data” as used herein, is a validation database, that automatically reviews edits, rules, omissions, standards of care, utilization guidelines, standards of care, authorization rules, relationships between HIPAA data code sets, including individual code database, source database, facility edits, coverage determinations for commercial and Medicare benefits, including self-funded benefit plans, against an expansive knowledgebase containing millions of government and industry rules and edits that cover health care claims.

“Liquidity partner” as used herein, finances or purchases secured receivables.

“adjudication cache” as used herein, are part of a network, the network is an interconnected group of adjudication caches.

“Map” or “Mappings” as used herein, are used to transform data and transfer it from a source data to a target database.

“Parent-Child” or “Parent and Child” or “Child and Parent” are database tables, but they are linked in a way that's described by a parent-child relationship. It's usually used to specify where one table's value refers to the value in another table (usually a primary key of another table).

“Price Matrix”, as used herein, is a price matrix database structure may offer flexible criteria to define and retrieve formulated internal threshold prices.

“Primary storage”, as used herein, is the collective methods and technologies used to capture and retain digital information that is in active use. Primary storage data is frequently accessed by applications or other hardware systems and users.

“Provider” or “health care provider” means an individual or entity duly licensed or legally authorized to provide health care services;

“Qualifying Circumstances” as used herein, defined various conditions that may reduce the user bids. Said conditions are defined by referenced data and source data.

“Reference data” “reference source data”, as used herein, reference data are sets of conditions, of values or classification schemas, source data that are referred to by systems, applications, data stores, processes, and reports, as well as by transactional and master records.

“Resource scheduling module” or “promotional scheduling” as used herein, is a flexible, configurable module that adapts to the health care providers staffing needs and it allows the health care provider to readjust their whole price higher or lower it based on the availability of resources. For instance, the health care provider can lower wholesale prices for any given day or range of days or time slot where there may a low volume of patients. The health care provider can increase their competitive position using the promotional scheduling module.

“Data Sources” or “Reference Data Sources”, as used herein, should be based on American Medical Association (AMA) CPT® guidelines, CMS guidelines, Correct Coding initiatives, commercial, Medicare, and all HIPAA data code sets including guidance, medical societies, and other widely accepted third-party sources. All guidelines change over time, source edits and rules should be kept up to date.

“Streaming data architecture”, as used herein, is an information technology framework that puts the focus on processing data in motion and treats extract-transform-load (ETL) batch processing as just one more event in a continuous stream of events.

“Target data” as used herein, can be source data or a target for data.

“Translation engine”, as used herein, shall include systems or methods that may involve a translation engine or other means for performing translation operations or other operations on user data, data, transactional data, remote data, one or more databases, in-memory databases, primary or secondary databases, including, but not limited to metadata. The translation operations or other operations may involve the translation of data or metadata from one or more formats, languages and/or data models to one or more formats, languages and/or data models.

“User” or “Users” or “consumers” or “bidder” or “bidder's” or ‘bidders” as used herein may be an individual, entity, corporation, employer, employee, uninsured, under-insured, insured, third party administrator, established patient, new user, or a benefit plan are bidding for one or more health care services.

“Wholesale price” as used herein, is a health care provider defined range of health care services with a range of health care service pricing, which are sold directly to the invention via an executed contract or agreement.

[In the following description, for purposes of explanation, numerous examples and specific details are set forth in order to provide a thorough understanding of the present disclosure. It will be evident, however, to one skilled in the art that the present disclosure as defined by the claims may include some or all of the features in these examples alone or in combination with other features in described below, and may further include modifications and equivalents of the features and concepts described herein.

For purposes of explanation, numerous examples and specific details are set forth in or to provide a thorough understanding of the present disclosure. In the event, however, to one skilled in the art that the present disclosure as defined by the claims may include some or all of the features in these examples alone or in combination with other features described below and may further include modifications and equivalents of the features and concepts described herein.
A. Reference source data “Add-on-Codes” A method of separately identifying a health care service that is performed in certain situations as an additional service on a graphical user interface (GUI) of a computer system, the method comprising:
   1. Receiving via the GUI, a user selection of a health care service and bid
   2. Matching a user syntax—selection to data targets
   3. Transforming, at the data-stage, a user selection of one or more health care services into one or more HIPAA code sets
   4. Identifying, a user selected health care services and bids that list separately in addition to the user's primary health care service
   5. Determining, by a processor, the Add-on-health care service and CPT code, and
   6. Designating that the separate health care service is an addition cost associated with one or more of the user's selection of health care services with CPT codes and corresponding bid,
   7. Enabling the user via GUI, to access from a widget a comparative shopping window,
   8. Transforming listed health care providers from the provider and supplier inventory,
   9. Combining the CPT codes to the appropriate six major subsections mentioned above
   10. Joining one or more CPT code, to subsection, to medical specialty,
   11. Targeting health care provider inventory data to match those health care providers who qualify by specialty to render the health care service(s) selected by the user
   12. Arranging the comparative shopping window by health care provider(s), health care provider prevailing charge for the health care service(s), whereby,
   13. Providing mapping tools, i.e., radius on a map, to expand the search criteria, such as zip code to view from the web enabled computer device a broader range of market prices, comprising of comparative prices for health care services by state, city, zip code,
   14. Shopping for a one or more health care providers from the comparative shopping window
   15. Selecting one or more health care providers from the comparative shopping window, based on prevailing charges
   16. Receiving, via the GUI, a user bid for one or more health care service and corresponding CPT code(s), on a GUI of a computer system,
   17. Determining, via a processor, the price matrix module compares the user's bid price for one or more health care services with a formulated internal threshold price. If the user's bid is exceeding the formulated internal threshold price the financial transaction, then occurs at the user's bid price. If the user's bid is below the formulated internal threshold price, the invention will programmatical position on the GUI of a computer system a message that bid was too low and then the user is given the opportunity to submit an incremented bid after a certain pre-set time period and comparison with the formulated internal threshold price is done again.
   18. Determining, via a processor, a formulated threshold price above which the invention is willing to accept any bid from the user.
   19. Calculating, the difference between the user's bid and the invention generated formulated internal threshold price, further
   20. Calculating, the difference between the formulated internal threshold price and the user's designated health care provider(s) wholesale price(s)
   21. Executing financial transactions when the user's price is above the formulated threshold price, and/or when the formulated threshold price is above the provider's wholesale price.
   22. Adjudicating, successful bids for health care services
   23. Adjusting, bids based on qualifying circumstances
   24. itemizing health care services, with bids and bid adjustments, including explanations of reasons for adjustment
   25. Generating an invoice on the GUI of a computing system
   26. Issuing price alert notification on a GUI of a user's web enabled device
   27. Scheduling price alerts over a period, specifically from creation date of the bid to the scheduled date of service,
   28. Determining, via a processor lower health care cost opportunity, such as having a surgery at an ambulatory surgical facility at significantly lower cost
   29. Automatically, sending scheduler reminder notices and or price alerts to remote computing devices
   30. Comparing and displaying site differentials between having health care services rendered at a office based surgical cache to having the surgery rendered at an ambulatory surgical facility to having a surgery at an acute care hospital, on a GUI of a computing system
   31. Sending scheduled health care services to designated health care provider and issuing a certificate of authorization, wherein confirming the scheduled appointment and confirming funding for scheduled health care services
   32. Receiving, via the GUI of a computer system, a confirmation of scheduled visit for the scheduled date of service
   33. Receiving, via the GUI of a computer system, a EDI 835 transaction or paper invoice for the completion of the health care service
   34. Reconciling, health care provider EDI 835 or paper invoice, against the user's winning bids
   35. Adjudicating the invoice
   36. Forwarding, approved line item health care services registered on the invoice to liquidity partner
   37. Funding, health care provider for delivery of health care services
   38. Receiving, an EDI 837 remittance statement from EDI partner, or
   39. Posting paper explanations of benefits to user financial history
   40. Reconciling funds in lockbox
   41. Forwarding insurance reimbursements to liquidity partner
   42. Managing, provider and contractual supplier terms and conditions, including wholesale price lists that are predefined by registered providers and suppliers, maintaining said contracts in inventory,
   43. Operating or maintaining an arrangement or contract with providers or suppliers relating to (A) health care services rendered by registrants, and (B) purchasing and paying for one or more health care services,
B. Reference source data designation “Separate Procedure” this statement in the CPT code database indicates that the health care service, while possible to be rendered separately, is generally included in a more comprehensive health care service, wherein the separate health care service received, via the GUI, from the user, programmatically, a bid would be unacceptable since a more comprehensive health care service is performed. In some embodiments, the “separate procedure” designation is used with codes in the surgery (CPT codes 10000-69999), radiology (CPT codes 70000-79999), and medicine (CPT codes 90000-99199) sections. When a database of related procedure from the same section, subsection, category, or subcategory is performed, a code with the designation of “separate procedure” is not to be purchased with the primary procedure. Whereby, reducing the cost of health care without compromising the user's health care needs.
C. Reference source data Laboratory panels: In a scenario, where the user is directed by their primary care physician to have some laboratory work done, from a web enabled device, the user searches and bids for the prescribed laboratory test; wherein receiving, via the GUI, a user selection of laboratory tests.
   Determining, by a processor, if the user selection of laboratory tests are components of a specific organ or disease-oriented laboratory panel (e.g., codes 80061 and 80059) or automated multi-channel tests (e.g., codes 80002-80019) if yes, programmatically, the invention will not accept a user bid separately, the adjudication manager will manipulate the data, wherein automatically rolling-up a less comprehensive laboratory test into the more one comprehensive panel or automated multichannel test code as appropriate that includes the multiple component tests. As part of a routine, the reference data will not allow the user to bid on an individual test that make up a panel or can be performed on an automated multi-channel test analyzer. Whereby, lowering the cost of care without compromising the quality of care.
D. Reference data source Mutually Exclusive Procedures; are health care services that are programmatically identified by the Adjudication Manager, whereby disallowing the user to make bids on mutually exclusive health care services on the scheduled date of service, because they are mutually exclusive of each other. Mutually exclusive health care services joined by CPT codes that cannot be rendered in the same session.
   Receiving, via the GUI, a user selection of one or more health care service, Adjudication Manager maps selected health care services to reference source data Determining, via a processor, a code pair match the mutually exclusive edits table
   Automating, a line-item rejection, whereby disallowing a user bid for the incidental health care service
   Transforming, wherein the Adjudication Manager rolls-up the incidental health care service, allowing the bid of the comprehensive health care service, whereby avoiding improperly maximizing of bids
E. A method of preventing improper bids on a GUI of a computer system, the method comprising of
   Receiving, via the GUI, a user selection of multiple health care services coupled with CPT codes, and corresponding bids;
   Determining, via a processor, qualifying circumstances and improper bids
   Pairing incorrect health care services combinations
   Rolling-up incidental health care service into a more comprehensive health care service
   Whereby preventing an improper bid when incorrect health care service combination is received, via the GUI.

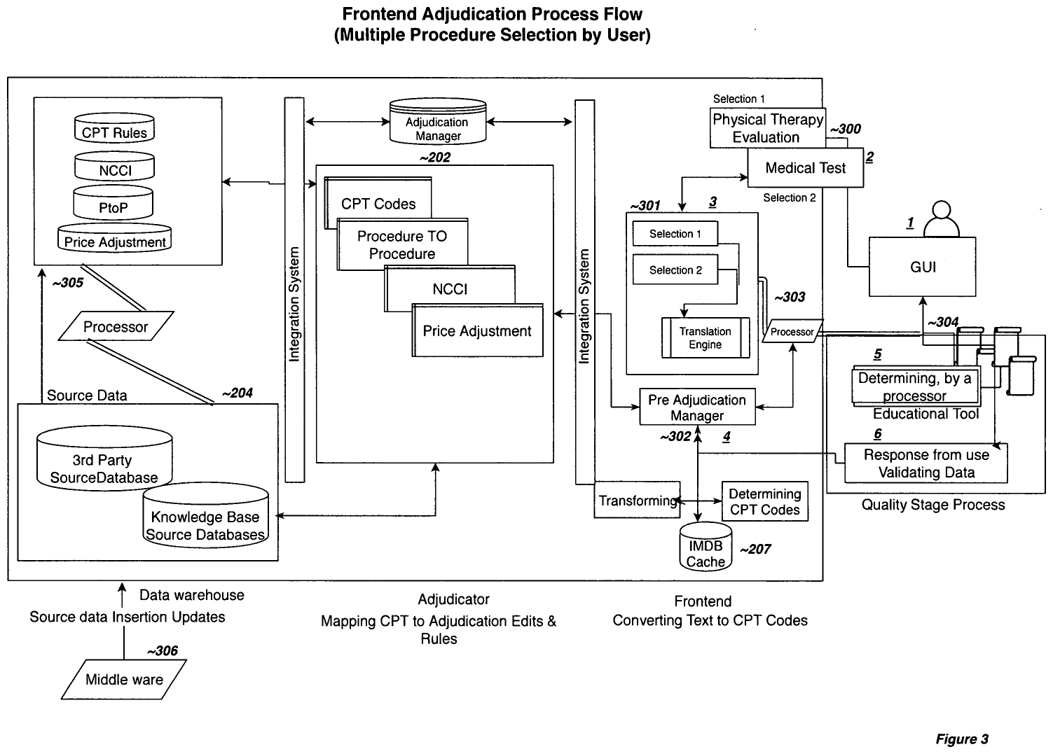

Process flow of avoiding improper bids on a GUI of a computer system FIG. 3
(1) User selects multiple health care services
(2) Adjudication Manager pairs health care services to reference data source same day of service
(3) All possible combinations of health care service pairs are matched to column one/column two national correct coding initiative reference data source
(4) Each row in the database represents a code pair
(5) Health care services identified in column 2 are determined to be incidental to column 1 health care services,
(6) A determination, via a process, column 3—indicators define qualifying circumstances
(7) An algorithm for indicator 0, will disallow a bid for and incorrect combinations if the combination of health care services are scheduled by the user on the same date of service
(8) An algorithm for indicator 1, makes an exception, allowing multiple bids for the same date of service
(9) An algorithm for indicator 9, code pairs are not subject to edit and rules
F. A method of adjusting multiple surgeries on a GUI of a computer system, the method comprising of:
   Receiving, via the GUI, a user selection of multiple surgeries,
   Transforming, user syntax queries to CPT codes
   Determining, by a processor, if standard multiple surgery edits and rules apply,
   Targeting multiple surgeries (indicator “2”)
   Ranking the surgeries linked to indicator 2 in descending order
   Base bids for each ranked surgery on the lower bid amount:
   100 percent of the bid amount for the highest valued surgery
   50 percent of the bid amount for the second highest valued surgery
   Determining, by a processor, if indicator “3” and multiple endoscopies are selected by the user.
      Example: After a pre-op consultation with a surgeon the surgeon's plan of care is documented and shared with the user/patient. The document indicates that the surgeon will perform a fiber optic colonoscopy (CPT code 45378).
   Receiving, via the GUI, a user selection a fiber optic colonoscopy, and a bid of $355.40
   Transforming, user syntax queries to CPT code 45378
   Displaying on a GUI a comparative shopping window
   Determining, via a processor, a price matrix module compares the user's bid price for one or more health care services with a formulated internal threshold price. If the user's bid is exceeding the formulated internal threshold price the financial transaction, then occurs at the user's bid price. If the user's bid is below the formulated internal threshold price, the invention will programmatical position on the GUI of a computer system a message that bid was too low and then the user is given the opportunity to submit an incremented bid after a certain pre-set time period and comparison with the formulated internal threshold price is done again.
   Determining, via a processor, a formulated internal threshold price above which the invention is willing to accept any bid from the user.
   Calculating, the difference between the user's bid and the invention generated formulated internal threshold price, further
   Calculating, the difference between the formulated internal threshold price and the user's designated health care provider(s) wholesale price(s)
   Executing financial transactions when the user's price is above the formulated internal threshold, and/or when the formulated threshold price is above the provider's wholesale price.
   Adjudicating, successful bids for health care services
   Adjusting, bids based on qualifying circumstances
   itemizing health care services, with bids and bid adjustments, including explanations of reasons for adjustment
   Generating an invoice on the GUI of a computing system
   Issuing price alert notification on a GUI of a user's web enabled device
   Scheduling price alerts over a period, specifically from creation date of the bid to the scheduled date of service,
   Determining, via a processor lower health care cost opportunity, such as having a surgery at an ambulatory surgical facility at significantly lower cost
   Automatically, sending scheduler reminder notices and or price alerts to remote computing devices
   Comparing and displaying site differentials between having health care services rendered at a office based surgical cache to having the surgery rendered at an ambulatory surgical facility to having a surgery at an acute care hospital, on a GUI of a computing system
   Sending scheduled health care services to designated health care provider and issuing a certificate of authorization, wherein confirming the scheduled appointment and confirming funding for scheduled health care services
   Receiving, via the GUI of a computer system, a remote health care provider confirmation of scheduled visit for the scheduled date of service
   Receiving, via the GUI of a computer system, an EDI 837 transaction or paper invoice for the completion of the health care service
   Reconciling, health care provider EDI 837 or paper invoice, against the user's winning bids
   Determining, by a processor, multiple endoscopies are submitted for purchase,
   Automatically, routing additional endoscopic services to reference source data in inventory
   Appending wholesale reserve price from health care provider's agreement to additional endoscopic services
   Determining, by a processor, a physician performed a biopsy on a lesion (code 45380) and removes a polyp (code 45385) from a different part of the colon. The physician's invoice lists codes 43580 and 45385. The adjudication manager module determines that the reserve value of codes 45380 and 45385 have the reserve value of the diagnostic colonoscopy (CPT code 45378) built in. Rather than calculating the funding requirements at 100 percent of the highest reserve value surgery (45385) and 50 percent for the biopsy on a lesion (CPT code 45380), fund the full value of the higher valued endoscopy (45385), plus calculate the difference between the next highest reserve value for an endoscopy (CPT code 45380) and the base endoscopy, fiber optic colonoscopy (CPT code 45378).
   Assuming the following physician's wholesale reserve price:
   45378—$255.40
   45380—$285.98
   45385—$374.56
   Calculating, the full wholesale reserve price of 45385 ($374.56), plus the difference between 45380 and 45378 ($30.58) for a total of $405.14.
   Reconciling the difference between the user selection and purchase price for a fiber optic colonoscopy (CPT code 45378) $355 and the total wholesale reserve price of $405.14 leaving a balance due from user of $150.14.
   Subtracting the outstanding balance $150.14 from the total reserve price of $915.94, determining, via a processor a net saving of $765.80 dollars; whereby, reducing the health care cost for multiple endoscopic treatments by $765.80, without compromising the quality of care.
   Forwarding to a third-party vendor, a mailing service, an itemize list of health care services, with easily to understand explanations of reasons for adjustment, said invoice statement includes the outstanding balance of $150.14 and the total net savings of $765.80 to the user.
   Adjudicating the bid for a fiber optic colonoscopy (CPT code 45378)
   Forwarding, approved line item health care services registered on the invoice to liquidity partner
   Funding, health care provider for delivery of health care services
   Receiving, an EDI 835 remittance statement from EDI partner, or
   Posting paper explanations of benefits to user financial history
   Reconciling funds in lockbox
   Forwarding insurance reimbursements to liquidity partner
   Managing, provider and contractual supplier terms and conditions, including wholesale price lists that are predefined by registered providers and suppliers, maintaining said contracts in inventory,
   Operating or maintaining an arrangement or contract with providers or suppliers relating to (A) health care services rendered by registrants, and (B) purchasing and paying for one or more health care services,
G. A method of identifying health care services, surgeries, with a high risk of hidden health care costs on a graphical user interface (GUI) of a computer system, the method comprising;
   Receiving, via the GUI, a user selection for a surgery
   Transforming, user syntax describing surgery, wherein Adjudication Manager retrieves from database management system matching syntax coupled with CPT code
   Displaying on the landing page, using widgets, on the GUI, educational tools related to descriptions of health care services, clinical indications, a crosswalk mechanism is used to link health care services to symptoms, conditions, disease,
   Targeting data with matching CPT codes
   Extracting edits and rules associated with CPT codes from the reference source data
   Accessing indicators within reference source data to determine, via a processor any qualifying circumstance
   Identifying a surgical procedure that can be performed by two surgeons or a team of surgeons
   Access reference source data to determine the bid amount for the surgery;
      If the user's bid for a surgery contains an indicator “0” the bid adjustment rules for two or team surgeons do not apply,
      The full value of the user's bid remains unadjusted or at 100% of the bid unless other adjustment rules apply;
      If the user's bid for a surgery contains an indicator “1”, base funding for bid for each physician at 62.5 percent of the bid amount

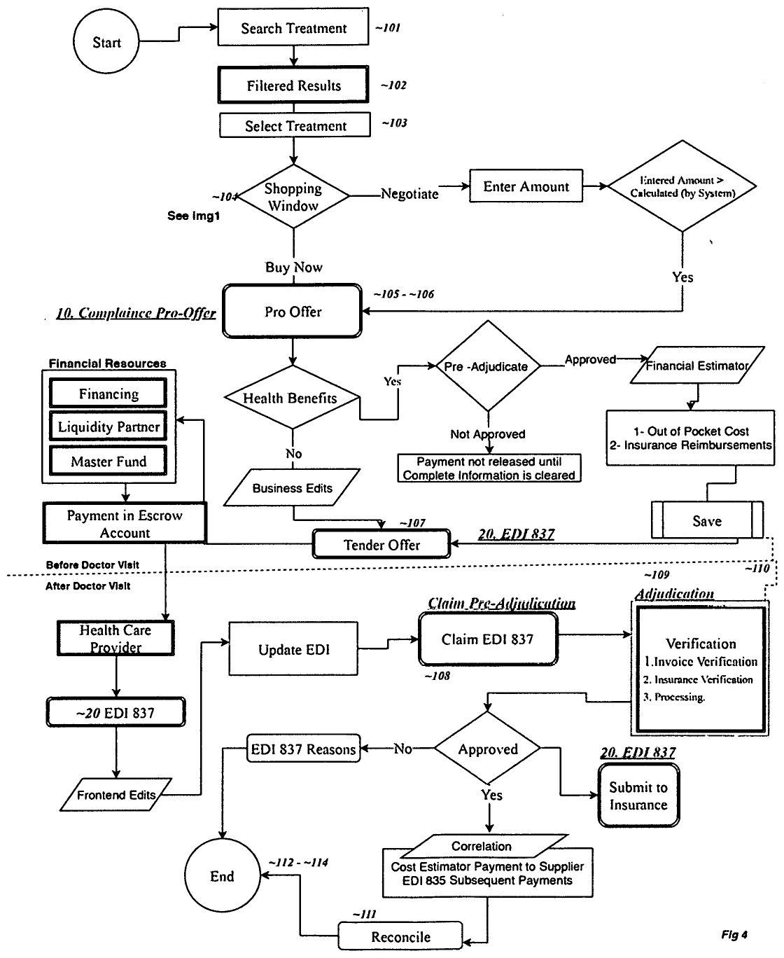

After receiving, via the GUI, a user selection for a surgery the adjudication manager identifies a surgical procedure which has a high likelihood of the surgery being performed by two surgeons or a team of surgeons.
Determining, by a processor, a price alter notification is generated and moved on the GUI of the computer system, wherein advising the user that a co-surgeon should be considered when making a bid.
Determining, by a processor, the price matrix adjusts the formulated internal threshold price to reflect 62.5 percent for each physician
Determining, via a processor, a formulated internal threshold price above which the invention is willing to accept any bid from the user.
Calculating, the difference between the user's bid and the invention generated formulated internal threshold price, further
Calculating, the difference between the threshold price and the user's designated health care provider(s) wholesale price(s)
Executing financial transactions when the user's price is above the formulated internal threshold, and/or when the formulated internal threshold price is above the provider's wholesale price.
Adjudicating, successful bids for health care services
Adjusting, bids based on qualifying circumstances
itemizing health care services, with bids and bid adjustments, including explanations of reasons for adjustment
Generating an invoice on the GUI of a computing system
Issuing price alert notification on a GUI of a user's web enabled device
Scheduling price alerts over a period, specifically from creation date of the bid to the scheduled date of service,
Determining, via a processor lower health care cost opportunity, such as having a surgery at an ambulatory surgical facility at significantly lower cost
Automatically, sending scheduler reminder notices and or price alerts to remote computing devices
Comparing and displaying site differentials between having health care services rendered at an office based surgical cache to having the surgery rendered at an ambulatory surgical facility to having a surgery at an acute care hospital, on a GUI of a computing system
Sending scheduled health care services to designated health care provider and issuing a certificate of authorization, wherein confirming the scheduled appointment and confirming funding for scheduled health care services
Receiving, via the GUI of a computer system, a confirmation of scheduled visit for the scheduled date of service
Receiving, via the GUI of a computer system, a EDI 835 transaction or paper invoice for the completion of the health care service
Reconciling, health care provider EDI 835 or paper invoice, against the user's winning bids
Adjudicating the invoice
Forwarding, approved line item health care services registered on the invoice to liquidity partner
Funding, health care provider for delivery of health care services
Receiving, an EDI 837 remittance statement from EDI partner, or
Posting paper explanations of benefits to user financial history
Reconciling funds in lockbox FIG. 4
Forwarding insurance reimbursements to liquidity partner
Managing, provider and contractual supplier terms and conditions, including wholesale price lists that are predefined by registered providers and suppliers, maintaining said contracts in inventory,
Operating or maintaining an arrangement or contract with providers or suppliers relating to (A) health care services rendered by registrants, and (B) purchasing and paying for one or more health care services,

Other Reference source data are included, but not limited to:
Pathology, laboratory; Automated Multi-channel test panels
IOM, Publication 100-04, Medicare Claims Processing Manual, Chapter 16, Section 90.1-90.3.1 This link will take you to an external website.
Diagnostic Cardiovascular Services
CMS Change Request (CR) 7442
Endoscopies (Multiple)
IOM, Publication 100-04, Medicare Processing Manual, Chapter 12, Section 40.6C.13
Multiple Procedure Payment Reduction (MPPR)
IOM, Publication 100-04, Medicare Claims Processing Manual, Chapter 5, Section 10.7

### Multiple Surgery Rules

IOM, Publication 100-04, Medicare Claims Processing Manual, Chapter 12, Section 40.6, Sub-Section
Physician Assistant-at-surgery Services
IOM, Publication 100-04, Medicare Processing Manual, Chapter 12, Section 110.1

Procedure to Procedure Code Edits (Column 1/Column 2)

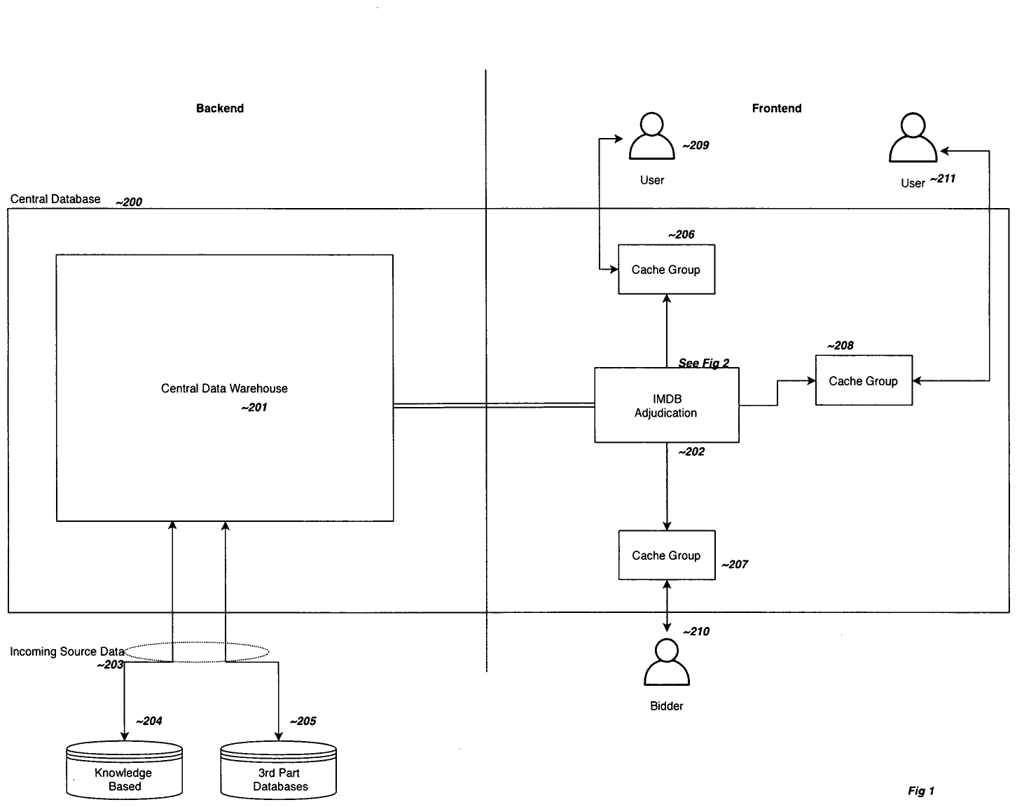

Turning now to FIG. 1, is a high level overview of a backend data integration system, with a central database 200 comprising a processor and a memory, receiving source data from a plurality of data sources in an enterprise 204,205; storing source data, as centrally managed data, in a data storage system configured with a plurality of data tables that comprise the data warehouse 201, including; using an extraction, translation, and load (ETL) tool to process 203 the source data and storing the processed data in the data warehouse 201; using processed source data to generate a plurality of enterprise data models, and storing the enterprise data models in the data warehouse 201; and, using the enterprise data models stored in the data warehouse 201 to generate a plurality of enterprise data views and storing the enterprise data views in the data warehouse 201. Assigning one or more enterprise data models including all reference data protocols, targeting edits and rules in the in-memory database adjudication (IMDBA) 202 database as reference data to the cache groups. The cache groups 209,210,211 does not include any persistent copies of the data that corresponds to the reference data; the front-end process further comprising performing query processing operations on the user data, including accessing the IMDBA 202 stored in data storage system that correspond to the reference data and operating on the accessed managed data and the local data in accordance with the query processing operations.

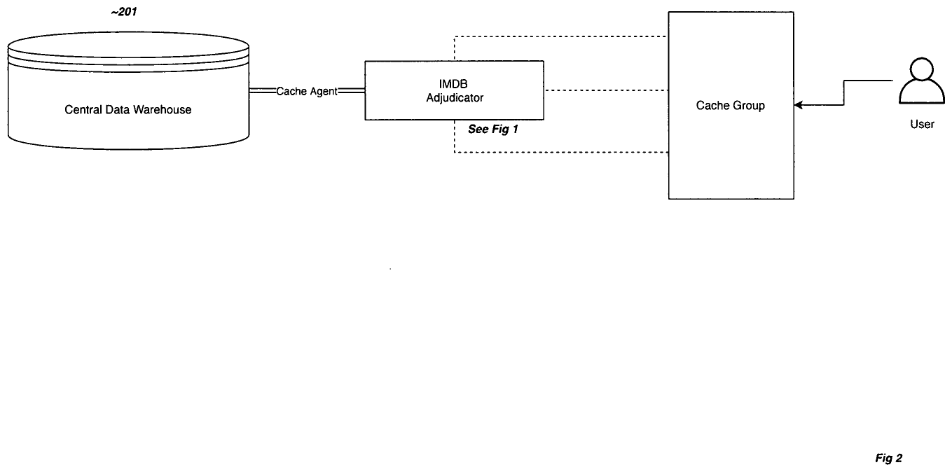

FIG. 2 is a high level overview, to illustrate a process used in the invention for receiving, via the GUI a user searching for one or more health care services and adjoining bid(s) from a widget positioned on the user's web enabled device landing page, computing input/output data, wherein communicating between one or more cache groups; and, another information processing system is communicating between, one or more cache groups and IMDBA; and, between the IMDBA and central database warehouse 201. There may be passthrough logic to retain user queries that are not subject to qualifying circumstance in a group cache. Likewise, queries that cannot be handled in the cache may be passed through to the IMDBA.

The IMDB cache maintains consistency between cache groups 208,209,210,211 and the central database warehouse 201 by propagating updates from cache groups 207,208,209 to the IMDBA and automatically refreshing data in cache group 207, 208, 209 from the IMDBA.

FIG. 3 illustrates a process flow of the adjudication or a health care service(s) and computing, a process used in the invention for receiving, via the GUI a user searching for one or more health care services and adjoining bid(s) from a widget positioned on the user's web enabled device landing page, computing input/output data, wherein communicating between one or more cache groups; and, another information processing system is communicating between, one or more cache groups and IMDBA; and, between the IMDBA and central database warehouse 201.

FIG. 2, and FIG. 3 represents a is a high-level diagram in FIG. 2 proceeded by FIG. 3 which is a more detailed version of FIG. 2; FIG. 3 illustrates a process flow of one reference data protocol, procedure to procedure unbundling edits, that may be assigned to cache group “medicine”.

The steps in FIG. 3 are described as follows: Step 1. The user sends an input from a widget on their landing page from their web enabled device for processing 300, and Step 2, the initial input from the a widget on the landing page is a semantic search for physical therapy, selection 1 and muscle testing, selection; Step 3, the streaming processor, extracts-transforms-loads (ETL), triggering batch processing as just one more event in a continuous stream of events; Step 4. The streaming processor may be used to query continuous data streams and to detect conditions like procedure to procedure, FIG. 3, quickly. The internal filter query will produce an event in the resulting stream immediately when the event matches the filter, as illustrated at Step 4. Between 301 and 302 a stream processor receives and sends data streams and executes the application; the Adjudication Manager 302 translates high-level queries into low-level expressions, creating a trigger statement to target source data where the expression “Physical Therapy” and “Muscle Testing” match. The filtering query finds a match and produces a sequence of events: the streaming processor creates a table, where the primary key is the CPT code, and the other columns and elements are linked as follows: the semantic phrases Physical Therapy and Muscle Test are in subsection “Medicine” in subsection Medicine a subset matches the phrase physical therapy evaluation and muscle testing, are mapped to “Physical Therapy” and “Muscle Testing and Range of Motion” header; ETL processing resulted in linking to another subset under Physical Therapy “Physical Therapy Evaluation” and the Physical Therapy subset physical therapy evaluation resulted in the development of another table with multiple rows of primary keys: 97161 through 97163 and another subset under Muscle Testing and Range of Motion was identified as muscle testing with the primary key CPT code and multiple CPT codes listed in rows under the primary key column and label as muscle test with the primary key including multiple rows of CPT codes ranging from 95831 through 95831.

CPT codes may be grouped and viewable by CPT code or subsection “Medicine” and a child of “Medicine” Physical Therapy included CPT code ranges 97161 through 97163; and the other child of “Medicine” “Muscle Testing and Range of Motion” were parsed, wherein grouping only those CPT codes under “Muscle Testing” 95831 through 95834. The output to the user selection 1 and selection 2, may be received, via the GUI, from the user's web enabled device, the user may view one or more educational tools, such as a listing of the CPT codes, with a widget positioned next to a row of one or more CPT codes; the user may view 304 the headings on the electronic page in plain language, such as a description of each CPT code, and column headers such as subsection, symptoms, conditions, diseases, medical specialty, including descriptions of health care services, including definitions like clinical terms. At the end of each row, a widget may be used to provide the option of printing out a sheet and/or conversing live with a user advocate. A push notification is sent to select from the Physical Therapy table and/or Muscle Test and Range of Motion table a CPT code.

For purposes of explanation, numerous examples and specific details are set forth in or to provide a thorough understanding of the present disclosure. In the event, however, to one skilled in the art that the present disclosure as defined by the claims may include some or all of the features in these examples alone or in combination with other features described below and may further include modifications and equivalents of the features and concepts described herein.

FIG. 3 shows “NCCI” and “Price Adjustment” these are root tables that have been updated by the INMDA, as mentioned above; the streaming processor triggered a command at 302 the CPT codes were split into two columns; Muscle testing CPT codes were place in column 1 and compared to physical therapy evaluation CPT codes in column 2, the query filer matched these two classes of health care services into pairs that matched the NCCI database. IMDBA created a new table with this pair and the description of the reference data rule, and the determination, via a processor, to adjust the selection of muscle testing down to zero value, whereby rolling-up column 2 into column 1, and only allowing the bid for a CPT code in column 1.

The adjudication manager 202 is a type of analytic database that is designed to streamline processing queries. It is part of a multicomputer system front-end system with multiple processor that are connected, target reference registries, identify qualifying and non-qualify circumstances. Where each processor 200 has its own memory and it is accessible by that processor and those processors can communicate with each other via an interconnect network.

The adjudication manager defines the transformation 202, for instance, taking a primary key and coupling primary keys to reference data, and joining date sensitive and non-data sensitive protocols, appending reference data and qualifying indicators to qualifying circumstances, connecting streaming processors to source data 306, testing transactional repository against knowledgebase databases 204, building data models 305, update IMDBA 202

In some embodiments, receiving, via a graphical user interface (GUI) of a computer, one or more user selections of health care service(s) with an adjoining user bid(s), an automated process takes the user data, extracts the information required for adjudication, transforms it into a format that can serve its business needs, and loads it to a centralized data repository, such as a centralized data warehouse and/or an in-memory-database management system. embodiments include, a streaming data architecture provides the means for receiving and sending data streams and executes system applications with real-time analytics logic. Upon receiving a user query, the stream processor reacts in real- or near real-time and triggers an action, such as remembering the process event for future reference.

The following tables exemplify the value proposition of this invention FIG. 3 process flow and FIG. 4 shows a detailed flow of the overall invention.

TABLE 1
Sample Database Structure

| | Column 2 | |
|---|---|---|
| | n = don't bill | |
| | the code in | |
| Column [symbol] | combination | Description of |
| Primary | with code in | Column 1 CPT |
| CPT Code | column 1 | Code |
| 97161 | 95831n; 95832n; | PT evaluation: |
| | 95833n; 95834n; | |
| 97162 | 95831n; 95832n; | PT evaluation: |
| | 95833n; 95834n; | |
| 97163 | 95831n; 95832n; | PT evaluation: |
| | 95833n; 95834n; | |

TABLE 3
PRICE MATRIX
Source of historical data -
Central database Warehouse 201 and IMDBA 202, 206, 207, 208, 209

| | Example | | | | |
|---|---|---|---|---|---|
| | of | | | | |
| Primary | National | | | | |
| Key | Avg. | | | | |
| Column 1 | Bids | 95831 | 98832 | 95833 | 95834 |
| 97161 | $86.49 | $33.16 | $32.80 | $43.52 | $56.58 |
| 97162 | $86.49 | $33.16 | $32.80 | $43.52 | $56.58 |
| 97163 | $86.49 | $33.16 | $32.80 | $43.52 | $56.58 |

TABLE 4
FIG. 3 Net Impact of Adjudication of Health Care Service Bids

| | 97161 | 95831 | Total Bid Price |
|---|---|---|---|
| Prior Art | $33.16 | $86.49 | $119.65 |
| Invention | $86.49 | | $86.49 |
| Dollars Saved by User | | | $33.16 |
| Net Savings to User | | | 28% |

What's claimed:

1. A method of joining source data protocols and financial transaction on a graphical user interface (GUI) of a computer system, the method comprising:
Converting a bidding context to one or more HIPAA data set codes, and automatically:
Translating data streams to one or more HIPAA data set codes
Targeting reference data to identify qualifying circumstances
Determining, by a processor, qualifying circumstances
Adjudicating one or more qualified conditions based on reference data protocols
Adjusting bid(s) that match qualifying circumstances
Automatically, listing selected health care services with preadjudicated bids amounts on to a widget at the landing page of a web enabled device, on the GUI of the computer system, based on qualifying circumstances and bid reduction dollar amounts, with easy to understand explanations for the determination; whereby lowering the price of the original bid, whereby
Mitigating the user's risk of over bidding

2. The method of claim 1, searching for a health care service from a widget positioned on a landing page on a graphical user interface (GUI) of a computer system, the method comprising of,
Inputting a description of semantic search by user of one or more health care services
Translating the input search criteria to one or more HIPAA data set codes
Determining, by a processor, procedural and diagnostic codes
Identifying target reference data
Adjudicating one or more qualified conditions based on reference data protocols
Adjusting bid(s) that match qualifying circumstances
Automatically, listing selected health care services with preadjudicated bids amounts on to a widget at the landing page of a web enabled device, on the GUI of the computer system, based on qualifying circumstances and bid reduction dollar amounts, with easy to understand explanations for the determination; whereby lowering the price of the original bid, whereby
Mitigating the user's risk of over bidding.

3. The method of claim auctioning adjudicated bids, from a widget positioned on a landing page from a use web enabled device, on a graphical user interface (GUI) of a computer system, the method comprising;
Receiving, a bid, from a user for one or more health care services Comparing user bid(s) and scheduled dates of service to formulated internal threshold price and reference data qualifying events
Determining, by a processor if the user dollar amount of the bid is below or above the formulated internal threshold price
Pushing notifications to landing page of the web enabled device, when the bid is below the threshold price
Messaging, user when bid(s) are successful, above the internal threshold price Automatically, itemizing bidding history onto an invoice to a position on the user's web enabled device, on the GUI of the computer system based on successful and unsuccessful bids.

4. The method of claim 3 further comprising purchasing health care services from health care systems on a graphical user interface (GUI), the method comprising:
Receiving, via the GUI, a health care provider's request to register online, wherein the health care provider wants to sell health care services;
Searching for health care charges and expected reimbursement with the health care provider's locality
Comparing health care charges and patient out of pocket-costs to peers
Accessing insurance company net health care provider to peers
Formulating utilization rates and market
Registering health care provider sales price list for health care services
Determining, by a processor, differential pricing
Automatically, emailing, and mailing a hard copy of the health care provider agreement with negotiated sale prices exhibits, based on determining differential pricing.

5. A method of claim 4, scheduling resources on a GUI of a computer system, the method comprising;
Reviewing business analytics
Identifying under utilized time slots, days, months, and so on
Lowering the registered sale prices for one or more health care services
Creating promotional campaigns
Determining, by a processor, health care provider availability
Promoting health care providers on the GUI of the computer system based on sale price and health care provider availability.

6. A method of claim 5, expanding search criterion of health care services and health care provider charges on a GUI of a compute system, the method comprising of;
Comparing health care provider charges by zip code
Sorting health care provider profiles by user preference
Grouping health care providers by specialty
Determining, by a processor, comparative prices, health care services, health care provider by geographic location, and
Automatically, moving best deals and pricing of health care services, to a widget on the landing page of the device, on the GUI of the computer system based on health care provider charges.

7. The method of funding adjudicated health care service(s) bid(s) on a GUI of a computer system, the method comprising,
Adjudicating a bid
Securitizing the bid
Converting a bid to a marketable security
Determining, via a processor, the guarantors' ability to purchase one or more bids
Automatically, embedding the adjudicated financial transaction into a widget on the landing page of a web enabled device, on the GUI of the computer system based on the determination of the marketability of the bid(s).
